(19) United States
(12) Patent Application Publication
(10) Pub. No.: US 2007/0184463 A1
(43) Pub. Date: Aug. 9, 2007

(54) Microfluidic device for purifying a biological component using magnetic beads

(75) Inventors: Josh Molho, Fremont, CA (US); Pamela Foreman, Los Altos, CA (US)

Correspondence Address: CALIPER LIFE SCIENCES, INC., 605 FAIRCHILD DRIVE, MOUNTAIN VIEW, CA, 94043-2234, US

(73) Assignees: Caliper Life Sciences, Inc., Mountain View, CA (US); Canon U.S. Life Sciences, Inc., Rockville, MD (US)

(21) Appl. No.: 11/542,652

(22) Filed: Oct. 2, 2006

Related U.S. Application Data
(60) Provisional application No. 60/722,372, filed on Sep. 30, 2005.

(51) Int. Cl. C12Q 1/68; C12M 3/00

(52) U.S. Cl. 435/6; 435/287.2

(57) ABSTRACT

A method of purifying a biological component found in a biological sample by extracting the biological component from the biological sample. The method is performed using a microfluidic device having at least one well for receiving the biological sample and at least one channel for introducing and removing fluids. A plurality of magnetic beads having a factor with an affinity for the biological component is introduced to the well together with a suitable biological sample. The biological sample is manipulated to release the biological component in proximity to the magnetic beads which are then segregated within the well while removing the biological sample. An elution solution for the biological component is introduced to the well and the elution solution together with the biological component are withdrawn therefrom.

### TECHNICAL FIELD

The present invention relates to the isolation of a component of interest from a biological sample. More particularly, embodiments of the present invention are directed toward purifying and thus preparing a component of interest in a biological sample for further manipulation within a microfluidic device.

### BACKGROUND OF THE INVENTION

Microfluidics refers to a set of technologies involving the flow of fluids through channels having at least one linear interior dimension, such as depth or radius, of less than 1 mm. It is possible to create microscopic equivalents of bench-top laboratory equipment such as beakers, pipettes, incubators, electrophoresis chambers, and analytical instruments within the channels of a microfluidic device. Since it is also possible to combine the functions of several pieces of equipment on a single microfluidic device, a single microfluidic device can perform a complete analysis that would ordinarily require the use of several pieces of laboratory equipment. A microfluidic device designed to carry out a complete chemical or biochemical analyses is commonly referred to as a micro-Total Analysis System (μ-TAS) or a “lab-on-a chip.”

A lab-on-a-chip type microfluidic device, which can simply be referred to as a “chip,” is typically used as a replaceable component, like a cartridge or cassette, within an instrument. The chip and the instrument form a complete microfluidic system. The instrument can be designed to interface with microfluidic devices designed to perform different assays, giving the system broad functionality. For example, the commercially available Agilent 2100 Bioanalyzer system can be configured to interface with four different types of assays—namely DNA (deoxyribonucleic acid), RNA (ribonucleic acid), protein and cell assays—by simply placing the appropriate type of chip into the instrument.

In a typical microfluidic system, all of the microfluidic channels are in the interior of the chip. The instrument can interface with the chip by performing a variety of different functions: supplying the driving forces that propel fluid through the channels in the chip, monitoring and controlling conditions (e.g., temperature) within the chip, collecting signals emanating from the chip, introducing fluids into and extracting fluids out of the chip, and possibly many others. The instruments are typically computer controlled so that they can be programmed to interface with different types of chips and to interface with a particular chip in such a way as to carry out a desired analysis.

Microfluidic devices designed to carry out complex analyses will often have complicated networks of intersecting channels. Performing the desired assay on such chips will often involve separately controlling the flows through certain channels, and selectively directing flows from certain channels through channel intersections. Fluid flow through complex interconnected channel networks can be accomplished either by building microscopic pumps and valves into the chip or by applying a combination of driving forces to the channels. Examples of microfluidic devices with built-in pumps and valves are described in U.S. Pat. No. 6,408,878, which represents the work of Dr. Stephen Quake at the California Institute of Technology. Fluidigm Corporation of South San Francisco, Calif., is commercializing Dr. Quake's technology. The use of multiple electrical driving forces to control the flow through complicated networks of intersecting channels in a microfluidic device is described in U.S. Pat. No. 6,010,607, which represents the work Dr. J. Michael Ramsey performed while at Oak Ridge National Laboratories. The use of multiple pressure driving forces to control flow through complicated networks of intersecting channels in a microfluidic device is described in U.S. Pat. No. 6,915,679, which represents technology developed at Caliper Life Sciences, Inc. of Hopkinton, Mass. The use of multiple electrical or pressure driving forces to control flow in a chip eliminates the need to fabricate valves and pumps on the chip itself, thus simplifying chip design and lowering chip cost.

Lab-on-a-chip type microfluidic devices offer a variety of inherent advantages over conventional laboratory processes such as reduced consumption of sample and reagents, ease of automation, large surface-to-volume ratios, and relatively fast reaction times. Thus, microfluidic devices have the potential to perform diagnostic assays more quickly, reproducibly, and at a lower cost than conventional devices. The advantages of applying microfluidic technology to diagnostic applications were recognized early on in development of microfluidics. In U.S. Pat. No. 5,587,128, Drs. Peter Wilding and Larry Kricka from the University of Pennsylvania describe a number of microfluidic systems capable of performing complex diagnostic assays. For example, Wilding and Kricka describe microfluidic systems in which the steps of sample preparation, PCR (polymerase chain reaction) amplification, and analyte detection are carried out on a single chip.

For the most part, diagnostic systems based on microfluidic technology have failed to reach their potential, so only a few such systems are currently on the market. Two of the major shortcomings of current microfluidic diagnostic devices relate to cost and to difficulties in sample preparation. Issues related to cost arise because materials that are inexpensive to process into chips, such as many common polymers, are not necessarily chemically inert or optically transparent enough to be suitable for diagnostic applications. To address the cost issue, technology has been developed that allows microfluidic chips fabricated from more expensive materials to be reused, lowering the cost per use. See U.S. Published Application No. 2005/0019213. However, issues of cross-contamination from previously processed samples can arise. These issues would be completely eliminated if each chip were used only once, suggesting the best solution may be to overcome the limitations of currently available polymer materials so that a chip can be manufactured inexpensively enough to be disposed of after a single use.

Processing of raw biological samples such as blood or other bodily fluids in microfluidic devices can be problematic. For example, raw biological samples can clog the narrow channels in a microfluidic device, especially if beads are also present in the channels. Therefore, in prior art microfluidic devices, treatment of raw biological samples is often required prior to introducing the sample into the device. An improved microfluidic diagnostic system would be completely automated, allowing sample preparation to be performed by the system, fully automating the assays performed by the system.

Difficulties can also arise if the component of interest in the sample is present in a low concentration. Because of the small cross-sectional area of microfluidic channels, the volumetric flow rate of sample through a microfluidic channel is low. Thus, if a large volume of sample needs to be processed to extract an adequate amount of a low concentration sample, the extraction process can be very time consuming. Quite often genetic materials of interest are present in low concentrations in a raw biological sample, so the extraction of enough genetic material for PCR amplification from the sample within a microfluidic device can be extremely time consuming, sometimes taking several hours.

Commercially available magnetic beads have been used to extract a component of interest from a raw biological sample in macrofluidic systems such as test tubes, vials, and microtiter plates. The principle behind these sample purification systems is well established. The magnetic beads in the sample purification systems have a magnetic core that is coated with a ligand that specifically binds to the component of interest. Thus when a raw biological sample is poured into a well in a microtiter plate or a vial containing the beads, the component of interest adheres to the outside of the beads. Since the beads are magnetic, they can be held in place within the vial or well by the magnetic field generated by a permanent magnet or an electromagnet. Thus, the beads containing the component of interest can be retained in the vial or well while the unwanted portion of the sample is removed.

Magnetic bead sample purification kits are sold by a variety of vendors, such as the Dynal® Biotech division of Invitrogen. Dynal® Biotech markets a line of magnetic beads under the brand name Dynabeads DNA DIRECT™ that is capable of isolating PCR-ready DNA from a variety of raw biological samples, including blood, mouth wash, buccal scrapes, urine, bile, feces, cerebrospinal fluid, bone marrow, buffy coat, and frozen blood. Sample purification processes employing Dynal® Biotech's Dynabeads product are designed be carried out in a variety of standard sized tubes that are placed in specially adapted receptacles equipped with strong permanent magnets that hold the magnetic beads in place within the tubes.

Magnetic beads have also been used in conjunction with microfluidic devices. A recent review of applications of magnetic beads in microfluidic devices by M. A. M. Gijs shows that the most common way of using magnetic beads in microfluidic devices is to entrain the beads within fluid flowing through a channel in the device, and to capture a component of interest on the beads from the surrounding fluid. See M. A. M. Gijs, Magnetic bead handling on-chip: new opportunities for analytical applications, Microfluid Nanofluid (2004) 1:22-40. Once the component of interest is captured on the bead, the beads themselves are captured using a magnetic field. The captured beads are either moved to a region of the chip where the component of interest can be detected or where the component of interest can be released from the beads to undergo further processing. In another reference, PCT Publication No. WO 2004/078316, Gijs describes devices that employ either a permanent magnet or an electromagnet to capture and transport beads within a microfluidic device.

Although magnetic beads have been used within microfluidic devices to extract a component of interest from a sample, such extraction processes are subject to the previously described problems when the sample is a raw biological sample. Indeed, the presence of beads within a microfluidic channel further narrows the effective flow cross section of the channel, thus exacerbating the previously described issues arising from clogging and low volumetric flow rates. Also, the flow of a raw sample through microfluidic channels can be difficult to control, since the fluid properties of the raw sample are generally not known.

Liu et al. describe a device in which magnetic beads are used to extract DNA from a raw biological sample such as blood. Liu et al., Self-Contained, Fully Integrated Biochip for Sample Preparation, Polymerase Chain Reaction Amplification, and DNA Microarray Detection, Anal. Chem. 2004, 76, 1824-1831. In Liu, the beads are coated with a ligand that specifically adheres to a particular type of cell within the sample. The DNA extraction process in Liu starts off by mixing the magnetic beads with the raw biological sample and flowing the sample/bead mixture through channels in a “biochip device” to a chamber within the device where the beads are captured through the application of a magnetic field generated by a permanent magnet. Once in the chamber, the cells adhering to the beads undergo further processing steps that purify and extract the DNA in the cells. Liu overcomes the difficulties associated with flowing a raw sample through a microfluidic device through the use of microscopic pumps and valves.

It is thus an object of the present invention to employ microfluidic devices for the preparation of raw biological samples.

It is a further object of the present invention to provide methods of extracting a component of interest from a raw biological sample by employing magnetic beads within a microfluidic device.

It is yet a further object of the present invention that those methods address the problems of flowing a raw sample through a microfluidic device without the need to resort to complicated microfluidic systems employing microscopic pumps and valves.

These and further objects will be more readily appreciated when considering the following disclosure and appended claims.

### SUMMARY OF THE INVENTION

A method of extracting a component of interest in a raw biological sample is performed using a microfluidic device having at least one well for receiving the raw biological sample and at least one channel for introducing and removing fluids into and out of the well. A plurality of magnetic beads having a ligand with an affinity for the component of interest is introduced into the well together with the raw biological sample. The raw biological sample is manipulated to release the component of interest in proximity to the magnetic beads so that the component of interest can bind to the ligand on the magnetic beads. The magnetic beads are then retained within the well with a magnetic field while the supernatant portion of the biological sample is removed from the well. An elution solution capable of releasing the component from the beads is then introduced into the well. Finally, the elution solution containing the component of interest is directed into a channel in the microfluidic device.

### BRIEF DESCRIPTION OF THE FIGURES

FIG. 1 is a generic representation of a typical microfluidic device that can be used to carry out methods in accordance with the invention.

FIGS. 2A-2E show cover layers that may be used as components of a microfluidic device in accordance with the invention.

FIG. 3 is a cross-sectional view across the line A-A in FIG. 2A.

FIGS. 4A-4G represent the steps in an embodiment of the invention.

FIGS. 5A-5G represent the steps in a second embodiment of the invention.

FIGS. 6A-6D represent the steps in a third embodiment of the invention.

FIG. 7 is a top view of a microfluidic device in accordance with the invention.

### DETAILED DESCRIPTION OF THE INVENTION

As noted previously, embodiments of the present method are directed to extracting a component of interest from a raw biological sample with magnetic beads. Sample preparation processes in accordance with the invention take place in a microfluidic device.

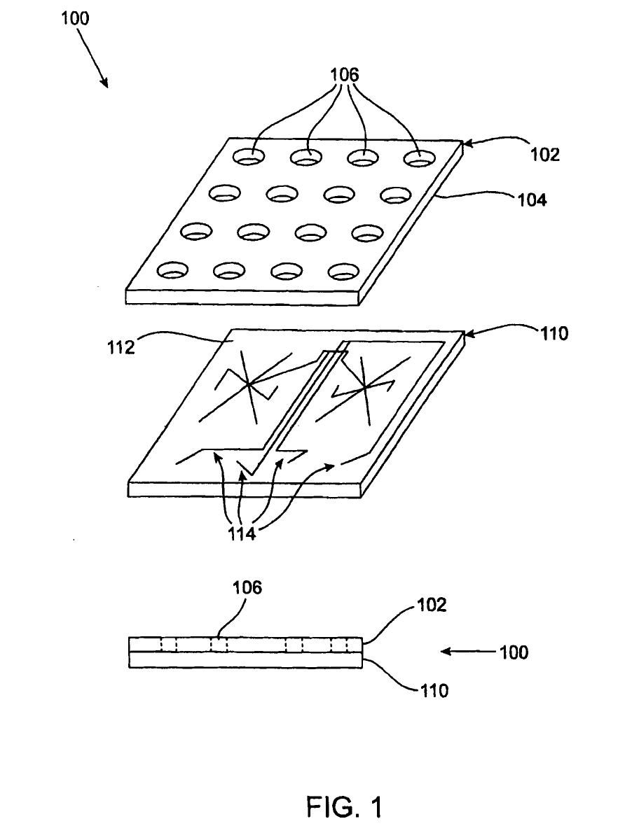

FIG. 1 is a generic representation of a typical microfluidic device that can be used to carry out methods in accordance with the invention. The top portion of FIG. 1 shows an exploded view of the device 100, which consists of two planar substrates 102,110; and the bottom portion of FIG. 1 shows a side view of the assembled device 100 after the two planar substrates 102,110 have been bonded together. Structures such as channels or chambers are formed within the interior of the assembled microfluidic device 100 by fabricating a pattern of grooves and trenches 114 on a surface 112 of one substrate 110 and bonding a corresponding surface 104 of the other substrate 102 onto the patterned surface 112. When the substrates are bonded together, the grooves and trenches 114 are enclosed, forming channels and chambers within the interior of the assembled device 100. Access to those channels and chambers is provided through ports 106, which are formed by fabricating holes in the upper substrate 102. The ports are positioned to communicate with specific points of the channels. For example, the ports 106 are positioned to communicate with the termini of the channels formed by enclosing grooves 114. The ports 106 can be used to introduce fluid into or extract fluids out of the channels of the device 100, or to allow driving forces such as electricity or pressure to be applied to the channels to control flow throughout the network of channels and chambers.

A variety of substrate materials may be employed to fabricate a microfluidic device such as device 100 in FIG. 1. Typically, since some structures such as the grooves or trenches will have a linear dimension of less than 1 mm, it is desirable that the substrate material be compatible with known microfabrication techniques such as photolithography, wet chemical etching, laser ablation, reactive ion etching (RIE), air abrasion techniques, injection molding, LIGA methods, metal electroforming, or embossing. Another factor to consider when selecting a substrate material is whether the material is compatible with the full range of conditions to which the microfluidic devices may be exposed, including extremes of pH, temperature, salt concentration, and application of electric fields. Yet another factor to consider is the surface properties of the material. Properties of the interior channel surfaces determine how these surfaces chemically interact with materials flowing through the channels, and those properties will also affect the amount of electroosmotic flow that will be generated if an electric field is applied across the length of the channel. Since the surface properties of the channel are so important, techniques have been developed to either chemically treat or coat the channel surfaces so that those surfaces have the desired properties. Examples of processes used to treat or coat the surfaces of microfluidic channels can be found in U.S. Pat. Nos. 5,885,470; 6,841,193; 6,409,900; and 6,509,059. Methods of bonding two substrates together to form a completed microfluidic device are also known in the art. See, for example, U.S. Pat. Nos. 6,425,972 and 6,555,067.

Materials normally associated with the semiconductor industry are often used as microfluidic substrates since microfabrication techniques for those materials are well established. Examples of those materials are glass, quartz, and silicon. In the case of semiconductive materials such as silicon, it will often be desirable to provide an insulating coating or layer, e.g., silicon oxide, over the substrate material, particularly in those applications where electric fields are to be applied to the device or its contents. The microfluidic devices employed in the Agilent Bioanalyzer 2100 system are fabricated from glass or quartz because of the ease of microfabricating those materials and because those materials are generally inert in relation to many biological compounds.

Microfluidic devices can also be fabricated from polymeric materials such as polymethylmethacrylate (PMMA), polycarbonate, polytetrafluoroethylene (TEFLON™), polyvinylchloride (PVC), polydimethylsiloxane (PDMS), polysulfone, polystyrene, polymethylpentene, polypropylene, polyethylene, polyvinylidine fluoride, ABS (acrylonitrile-butadiene-styrene copolymer), cyclic-olefin polymer (COP), and cyclic-olefin copolymer (COC). Such polymeric substrate materials are compatible with a number of the microfabrication techniques described above. Since microfluidic devices fabricated from polymeric substrates can be manufactured using low-cost, high-volume processes such as injection molding, polymer microfluidic devices could potentially be less expensive to manufacture than devices made using semiconductor fabrication technology. Nevertheless, there are some difficulties associated with the use of polymeric materials for microfluidic devices. For example, the surfaces of some polymers interact with biological materials, and some polymer materials are not completely transparent to the wavelengths of light used to excite or detect the fluorescent labels commonly used to monitor biochemical systems. So even though microfluidic devices may be fabricated from a variety of materials, there are tradeoffs associated with each material choice.

To perform methods in accordance with the invention, a plurality of magnetic beads is placed within a well in the microfluidic device. Within the context of this disclosure, a well is a fluid-containing reservoir that is connected to one or more of the channels within the interior of the device through a port. During operation of the microfluidic device, the wells serve as either a source of fluid to be introduced into the channel network or as a receptacle for fluid exiting the fluid network. Wells are typically accessible from the exterior of the chip.

Wells on microfluidic devices can be configured in a number of different ways. For example, in the microfluidic device shown in FIG. 1, the ports 106 themselves can function as wells. The volume of those wells 106 would be determined by the thickness of the top substrate layer 102 and by the diameter of the circular opening 106 forming the well. Typical glass substrates range in thickness from about 0.5-2 mm. So, for example, if the holes forming the ports 106 have a diameter ranging from about 0.5-3 mm, and the volume of the wells formed by the port openings would range from 0.1-15 μl. It is possible to form higher volume wells by attaching a cover layer to the microfluidic device so that apertures in the cover layer are aligned with the ports 106. Detailed descriptions of cover layers that can be used with microfluidic devices compatible with embodiments of the invention are provided in U.S. Pat. No. 6,251,343.

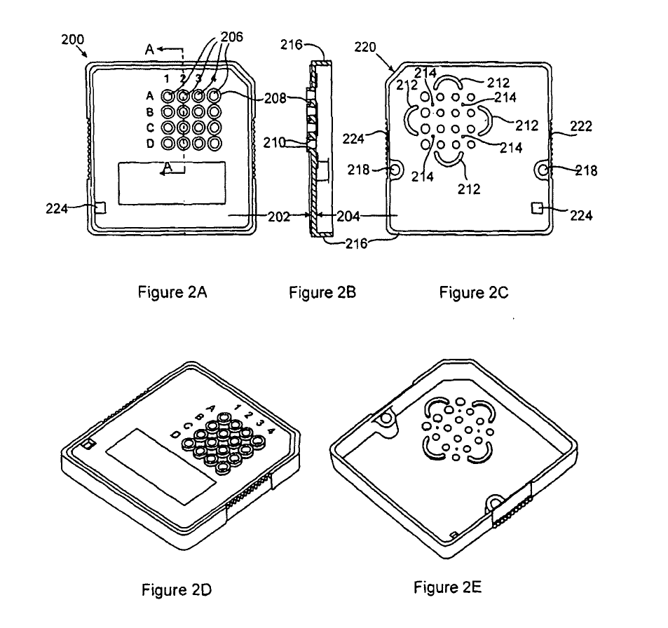

FIGS. 2A-2E show a cover layer 200 that can be used with the microfluidic device shown in FIG. 1. FIG. 2A is a top view, 2B a cross-sectional view, 2C an underside view, 2D a perspective view of the top side, and 2E a perspective view of the bottom side of the cover layer 200. The cover layer 200 is designed to receive the chip 100 in a mounting region on the underside of the cover layer 200 that is delineated by four ridges 212 that protrude from the underside of the cover layer.

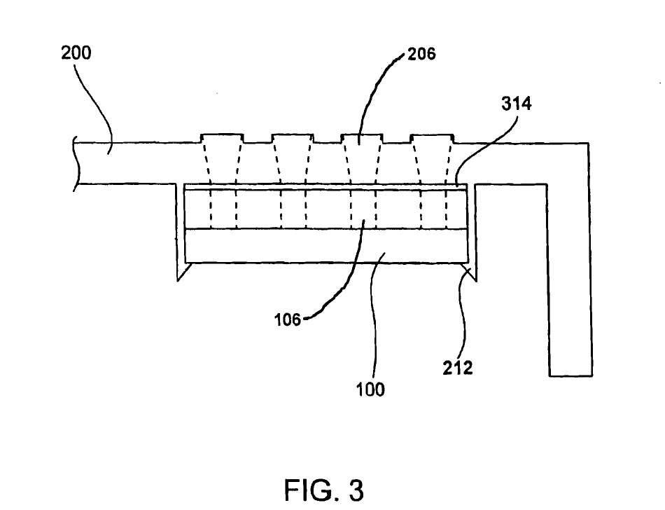

A cross-sectional view across the line A-A in FIG. 2A is shown in FIG. 3. In FIG. 3, a microfluidic device 100 is mounted onto the underside of a cover layer 200. It can be seen that the apertures 206 in the cover layer are aligned with the ports 106 in the microfluidic device, and the combination of each aperture 206 and port 106 forms a well with a total volume equal to the volume of the aperture and the volume of the port.

Methods in accordance with the invention can be practiced on a wide variety of microfluidic devices, not just the device shown in FIGS. 1-3. The defining characteristics of a microfluidic device that is compatible with the practice of the invention is simply that the device contains a well, and that flow into and out of the well can be controlled by an instrument that interfaces with the microfluidic device. So, for example, methods in accordance with the invention could be practiced on microfluidic devices formed from more than two substrates layers. Examples of such multilayer microfluidic devices can be found in U.S. Pat. Nos. 6,408,878 and 6,167,910. Also, although microfluidic devices compatible with the invention are typically substantially planar, the major surface of the microfluidic device does not have to be rectangular or square. An example of a round microfluidic device that could be compatible with embodiments of the invention is shown in U.S. Pat. No. 6,884,395.

The material from which the microfluidic device is made is largely irrelevant to the practice of the invention, as long as the material does not contaminate or otherwise interfere with the reagents, samples, or reactions involved in practicing the invention. Furthermore, details of the well structure, such as its cross-sectional shape, whether it is formed entirely within one substrate, in multiple substrates, or in a substrate and a cover layer, are largely irrelevant to the practice of the invention, as long as the well interfaces with a microfluidic channel network, and as long as the well is large enough to accommodate enough raw sample and magnetic beads to procure the desired amount of the component of interest. For example, if the well is formed from the combination of a port in a microfluidic device and an aperture in a cover layer, the aperture and port do not have to be the same shape, size, or depth, as long as the combination of the aperture and port define a volume capable of being used as a fluid reservoir.

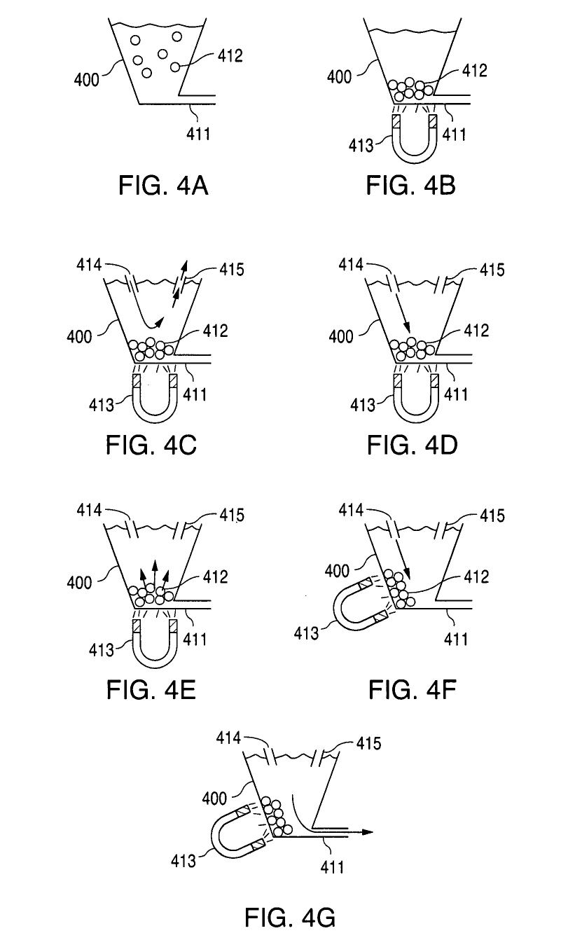

In providing a further appreciation of the present invention, reference is made to FIG. 4. Panels A-G of FIG. 4 represent a schematic cross-sectional view of a portion of a microfluidic device containing a well 400 in fluid communication with a channel 411 at various steps in a sample purification process in accordance with the invention. The microfluidic device must be interfaced with an instrument that permits control of the flow through channel 411. In certain embodiments, almost any methods of controlling the flow through microfluidic channels known in the art could be used to control the flow through channel 411. For example, the electrokinetic flow control methods described in U.S. Pat. No. 6,010,607; the pressure control methods described in U.S. Pat. No. 6,915,679; and the mechanical methods described in U.S. Pat. No. 6,408,878 are compatible with embodiments of the invention. As previously discussed, control of flow through the channels of the microfluidic device comprising well 400 would be directed by an instrument (not shown) that interfaces with the device. Regardless of the particular flow control system employed, the flow in channel 411 must be initially controlled so that fluid contained in well 400 does not flow into channel 411.

The purification process illustrated in FIG. 4 requires the addition of magnetic beads, and a number of reagents, to the sample. The magnetic beads are coated with a ligand that specifically binds to the component of interest in the sample. Methods of fabricating magnetic beads, and of coating the beads with ligands, are well known in the art. The reagents required to carry out a sample purification process with magnetic beads include a washing buffer that removes contaminants from the component of interest bound to the ligand on the beads, an elution buffer that releases the component of interest from the beads, and, in some cases, a lysing agent that releases genetic material from the interiors of cells in the sample.

Magnetic beads and the reagents required to carry out sample purification processes on a variety of different samples and components of interest are commercially available in kits. Such kits are sold by a variety of vendors, such as the Dynal® Biotech division of Invitrogen, Agencourt Bioscience Corporation (a wholly owned subsidiary of Beckman Coulter), Chemagen Biopolymer-Technologie AG (Germany), and Qiagen (Netherlands).

The following illustrative embodiments employ Dynal® Biotech's Dynabeads DNA DIRECT™ Universal product kit to extract DNA from a blood sample. This product was chosen because it is sold as a kit that contains all of the reagents required to carry out a sample purification process in accordance with the invention, and because the protocol implementing that process is a single-step protocol that does not involve a centrifugation step. Detailed protocols employing the Dynabeads DNA DIRECT™ Universal product are described in the Dynal® Biotech web site (www.dynalbiotech.com) and in the product literature that accompanies the DNA DIRECT™ Universal product. Dynal® Biotech also provides protocols for the DNA DIRECT™ Universal product that are capable of isolating PCR-ready DNA from a variety of raw biological samples, including mouth wash, buccal scrapes, urine, bile, feces, cerebrospinal fluid, bone marrow, buffy coat, and frozen blood. According to the product literature, the Dynabeads DNA DIRECT™ Universal product can extract enough DNA from a 30-μl blood sample to carry out 30-50 PCR amplifications. The product literature indicates that a workable amount of DNA can be extracted from a sample volume at least as low as 5 μl. The standard protocol for DNA extraction using Dynabeads calls for 200 μl of beads suspended in buffer. Naturally, the volume of the well must be large enough to accommodate not only the sample, but also the beads and the reagents used in the sample purification process. Accordingly, the wells in the embodiment shown in FIG. 4 would typically have a volume of at least around 250 μl. As one skilled in the art would recognize, for the type of microfluidic device structure shown in FIGS. 1-3, the well volume can be manipulated by changing the volume of the ports 106 by varying the size of the opening forming the port, or by varying the thickness of the top substrate 102, and/or by changing the volume of the apertures 206 in the cover layer by varying the size of the opening forming the aperture or by varying the thickness of the cover layer 200.

FIG. 4A represents the first step of the method in which a raw biological sample, a plurality of magnetic beads 412, and reagents are placed into well 400. The component of interest may be suspended within the biological component in such a way that it can interact with the surfaces of the beads, or it may be contained within biological structures such as cells which must be lysed before the component of interest can interact with the surfaces of the beads.

The reagents included in the DNA DIRECT™ Universal product kit include a lysing agent that can release genetic material such as DNA from the interior of a cell in a raw biological sample. The magnetic beads 412 are coated with a ligand, such as DNA complementary to the DNA that is the component of interest, that specifically binds to the component of interest. Ligand coatings for magnetic beads that specifically bind to a variety of different biological materials, including cells, DNA, mRNA, and proteins, are known in the art. Returning to FIG. 4A, DNA released from blood cells in the raw blood sample will adhere to the coating on the magnetic beads, thus extracting the DNA from the raw sample. The standard protocol for DNA extraction from blood using Dynabeads calls for the beads to be incubated with the sample at room temperature for 5 minutes. Agitation is not required during the incubation period.

After the required incubation period has transpired, a magnetic field is applied to the well in order to retain the magnetic beads 412 at the bottom of the well 400 as shown in FIG. 4B. The magnetic field can be generated by a permanent magnet or by an electromagnet. Permanent rare earth magnets, such as magnets fabricated from neodymium-iron-boron, can generate sufficiently strong magnetic force to retain the beads 412 at the bottom of the well 400. Devices with electromagnets capable of generating fields strong enough to retain or transport magnetic beads in a microfluidic device are also known in the art. See, e.g., PCT Publication Nos. WO 2004/078316 and WO 03/061835. The permanent magnet or electromagnet generating the magnetic field that retains the magnetic particles 412 at the bottom of the well 400 is schematically represented as magnet 413 in FIG. 4B.

Since the applied magnetic field retains the magnetic beads 412 at the bottom of well 400, fluid can be removed and added to the well without displacing the beads. Thus, the supernatant portion of the raw sample can be removed from the well 400, and wash buffer can be repeatedly added and removed from the well 400, to remove the unwanted portion of the raw sample so that only the component of interest bound to the beads remains. The fluid removal and addition steps are schematically represented in FIG. 4C.

In some embodiments, the fluid can be removed and added to the well using standard liquid handling equipment. Examples of commercially available automated liquid handling equipment that could be used in embodiments of the invention are the Genesis® and Freedom EVO products sold by the Tecan Group, Ltd. (Switzerland), and the Biomek® FX and Biomek® 2000 products sold by Beckman Coulter, Inc. (Fullerton, Calif.). In the embodiment shown in FIG. 4C, the instrument interfacing with the microfluidic device containing the well controls the flow of fluid through an inlet tube 414 and an outlet tube 415. As such, in the embodiment shown in FIG. 4C, a suitable wash buffer can be cycled through well 400 by introducing the wash buffer into the well 400 through inlet 414, and then withdrawing the wash buffer through outlet 415. Note that since the magnetic beads 412, which are bound to the component of interest, remain magnetically retained at the bottom of well 400, the beads 412 are not inadvertently swept out of well 400 during the cycling of wash buffer therethrough.

After undesired components of the raw sample have been removed from the well 400 by the wash buffer, the component of interest retained on the magnetic beads 412 can be eluted. Two alternative methods of introducing the elution buffer that releases the component of interest from the magnetic beads 412 are shown in FIGS. 4D and 4E. In FIG. 4D, the elution buffer is introduced into the well from outside the microfluidic device. As was the case with the wash buffer, the elution buffer could be introduced into well 400 with standard liquid handling equipment or, as specifically shown in FIG. 4D, through an inlet tube 414 whose flow is controlled by the instrument interfacing with the microfluidic device containing the well.

Alternatively, as represented in FIG. 4E, the elution buffer could be introduced through channel 411 into the well 400. In the embodiment of FIG. 4E, the elution buffer would be stored in another well (not shown) on the microfluidic device, and the instrument interfacing with the microfluidic device would direct flow from that well, through channel 411, into well 400. The conceptual embodiment shown in FIG. 4E is particularly appealing as elution buffer is caused to percolate through beads 412 as the beads are magnetically retained at the bottom of well 410.

To help the elution buffer release the maximum amount of the component bound to the beads, the beads can be agitated during the elution step. As shown in FIG. 4F, the beads can be agitated by moving the beads within the well by manipulating the magnetic field generated by magnet 413. For example, FIG. 4F schematically illustrates repositioning the magnet 413 generating the field so that the magnetic particles 412 are moved to one side of the well 412.

Under the standard Dynabead protocol, the time required to accomplish elution is on the order of 5 minutes. Once the elution is complete, the component of interest will be present in the elution buffer either in suspension or in solution. As shown in FIG. 4G, the elution buffer containing the component of interest can be directed into channel 411 by the flow control system in the instrument interfacing with the microfluidic device. Note that a magnetic field is still being applied to the magnetic beads 412, so the beads will be retained within the well 400. Once the fluid containing the component of interest is directed into channel 411, the flow control system can direct the fluid into other areas of the microfluidic device where it can undergo further processing steps such as PCR amplification and/or detection.

In an alternative embodiment, the elution steps shown in FIGS. 4F and 4G can be replaced by an elution process in which elution buffer is flowed under pressure into well 400, as shown in FIG. 4E, while an electric field is applied across the length of channel 411 that transports the inherently negatively charged DNA molecules eluted from the beads into channel 411 against the flow of elution buffer. This alternative elution process is based on the selective ion extraction technology disclosed in, for example, U.S. Published Patent Application No. 2003/0230486.

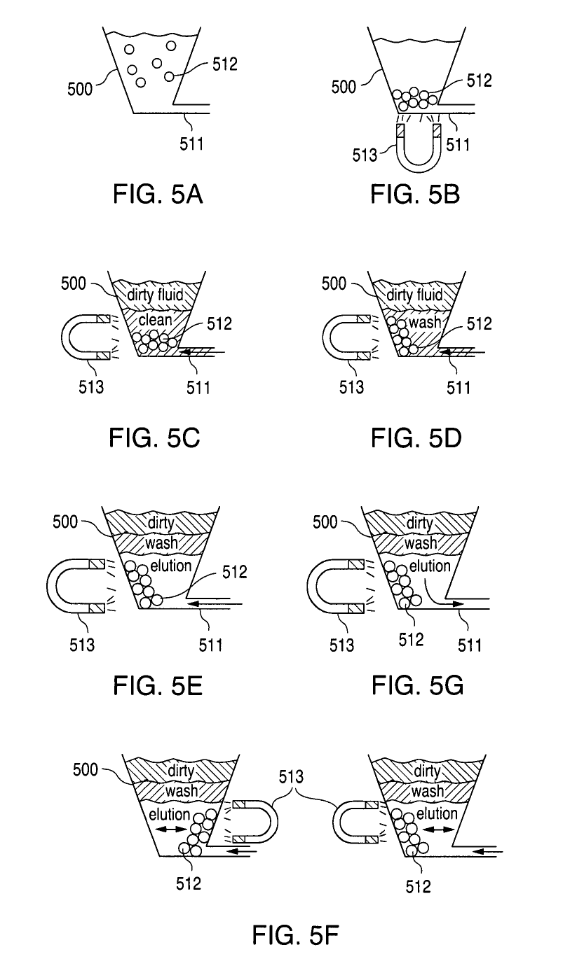

An alternative embodiment in which the wash buffer and elution buffer are introduced into the well through one or more microfluidic channels is shown in FIGS. 5A-5G. In the embodiments of FIGS. 5A-5G, a single channel 511 is connected both to a well containing wash buffer and to a well containing elution buffer. The initial situation shown in FIG. 5A is identical to the situation depicted in FIG. 4A: a raw sample and a suspension containing magnetic beads is introduced into well 500, while a flow control system maintains a zero flow rate through channel 511. Once again, in this example embodiment, the raw biological sample is blood, and the reagents and beads used to extract the component of interest (DNA) from the raw sample are the components of the commercially available Dynabeads DNA DIRECT™ Universal product kit. Thus, in this embodiment the magnetic beads 512 are suspended in a buffer containing a lysing agent.

After the appropriate incubation period, the magnetic beads 512 are subsequently retained at the bottom of well 500 in the same manner as shown in FIG. 5B. The step shown in FIG. 5B is essentially identical to the step represented by FIG. 4B in the previously described embodiment. The step represented in FIG. 5C, however, differs from the step shown in FIG. 4C. In FIG. 5C, wash buffer is introduced into well 500 through channel 511. This is accomplished by having the flow control system in the instrument (not shown) interfacing with the microfluidic device direct flow from a well containing wash buffer (not shown) through channel 511 into well 500. In contrast, in the previously described embodiments shown in FIG. 4C the wash buffer was introduced into well 500 from a source external to the microfluidic device. In the embodiment shown in FIG. 5C, where the wash fluid is introduced at the bottom of well 500, poor mixing between the supernatant portion of the raw sample and the wash buffer causes the supernatant sample to be displaced from the bottom of the well by the incoming wash buffer. As shown in FIG. 5C, a sufficient amount of wash buffer can be introduced into the well 500 so that the beads 512 at the bottom of the well 400 are completely immersed in wash buffer. At this point, it may be desirable to reposition the magnet 513 to manipulate the field applied to the beads so that the beads are agitated within the wash buffer. This agitation step, which is represented in FIG. 5D, can enhance the effectiveness of the wash buffer in removing unwanted portions of the raw sample from the vicinity of the beads 512.

As was the case in the embodiment shown in FIGS. 4A-4G, in the embodiment shown in FIGS. 5A-5G the wash step is followed by the introduction of an elution buffer. As shown in FIG. 5E, in the current embodiment the elution buffer is introduced through channel 511. This is accomplished by having the flow control system in the instrument (not shown) interfacing with the microfluidic device direct flow from a well containing elution buffer (not shown), through channel 511 into well 500. Once again, the poor mixing between the elution buffer and the wash buffer will cause the incoming elution buffer to displace the wash buffer from the bottom of well 500. FIG. 5E represents the situation in well 500 after a sufficient amount of elution buffer has been introduced into well 500 to displace the wash buffer from the vicinity of the beads 512. As shown in FIG. 5F, the beads can be agitated to increase exposure of the surfaces of the beads to the elution buffer. After the elution step is complete, the elution buffer containing the component of interest can be withdrawn from well 500 through channel 511 as shown in FIG. 5G.

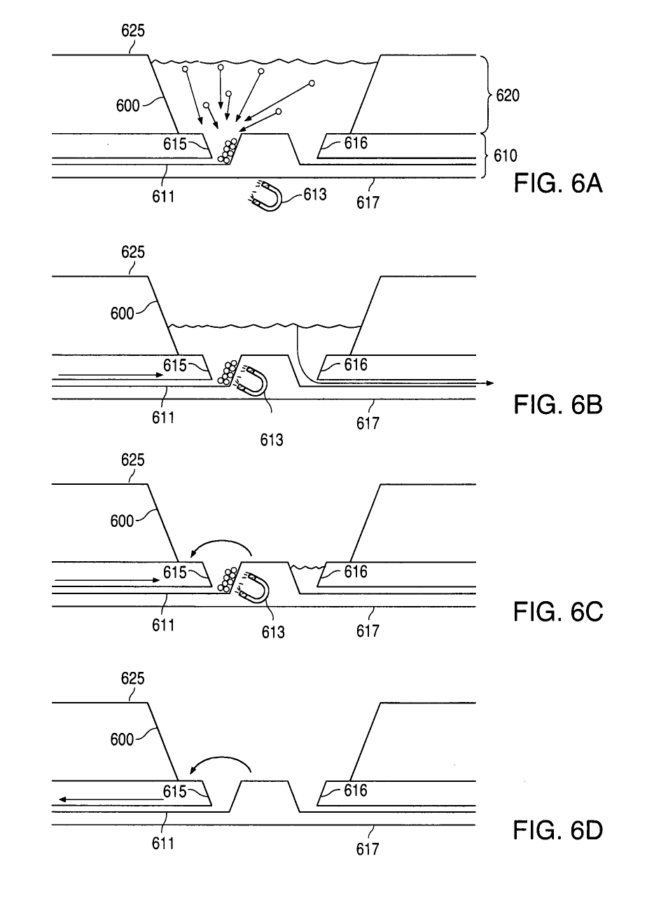

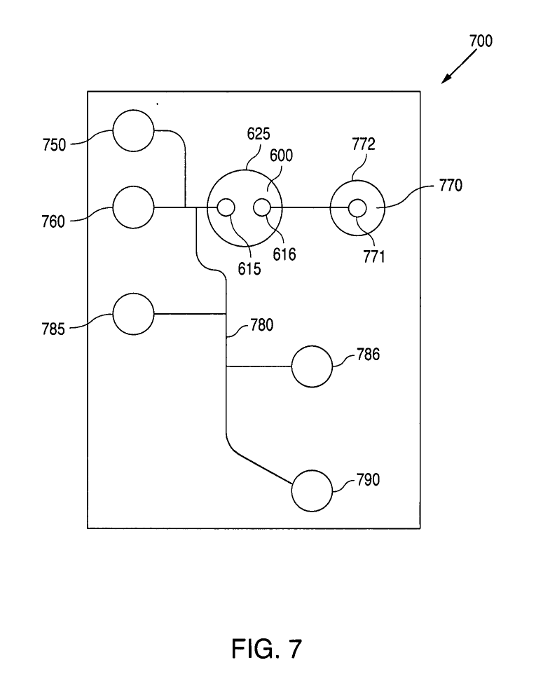

Not surprisingly, other variations on the present theme can be employed in carrying out this inventive method. A third embodiment of the invention is schematically represented in FIGS. 6A-6D and in FIG. 7. In this embodiment, well 600 consists of an aperture in a cover layer 620, which is bordered by an opening 625 in the top surface of the cover layer 620, and two ports 615 in the main body 610 of the microfluidic device encompassed by the aperture. A top view of the microfluidic device illustrated in FIGS. 6A-6D can be seen in FIG. 7, where the aperture opening 625 in the cover layer encompasses the two ports 615,616 in the underlying main body of the device. The steps in the embodiment in FIGS. 6A-6D are quite similar to the steps in the embodiment shown in FIGS. 5A-5G, with the main difference being that the well 600 in FIGS. 6A-6D is in fluid communication with two channels 611,617 instead of just one channel, e.g., 511. The presence of the second channel in the embodiment of FIGS. 6A-6D allows undesired material, such as supernatant sample and used wash buffer, to be removed from the well 600.

FIG. 6A represents the application of a magnetic field by a magnet 613 to collect the magnetic beads 612 within one of the ports 615 after the magnetic beads 612 have been incubated with the raw sample solution so that the cells in the raw sample are lysed to release the component of interest from the cells, and so the released component of interest can then bind to the ligands on the surface of the magnetic beads. As previously discussed, if a commercially available magnetic bead kit is employed, the standard conditions for lysing and binding specified for the kit can be used.

As shown in FIG. 6B, after the beads are retained within the portion of the well 600 defined by port 615, wash buffer can be introduced into the well through channel 611 and withdrawn from the well 600 through channel 617. Withdrawal of the used wash buffer from the well 600 should aid in the removal of undesired material from the vicinity of the beads 612.

After the washing step in FIG. 6B is complete, elution solution can be introduced through channel 611 as shown in FIG. 6C. As shown in FIG. 7, channel 611 is in fluid communication with a well 750 that contains wash buffer and a well 760 that contains elution buffer. Known methods of controlling flow in a microfluidic device can be used to selectively direct flow from either well 750 or well 760 through channel 611 into well 600. In the embodiment shown in FIG. 7, fluid withdrawn from well 600 through channel 617 can be directed by a flow control system into a waste well consisting of port 771 and aperture 772.

After the required incubation period for elution has transpired, the elution buffer containing the component of interest can be withdrawn from well 600 through channel 611 as shown in FIG. 6D. As schematically illustrated in FIG. 7, flow from channel 611 can be directed into channel 780, where the component of interest can be subjected to further processing. For example, wells 785 and 786 could contain reagents that will react with the component of interest as it travels through channel 780 towards waste well 790.

When the component of interest is genetic material such as DNA, the further processing that takes place after sample purification will often include PCR amplification of the DNA. So, for example, the PCR process described in U.S. Published Patent Application No. 2002/0197630 could be performed on a sample purified using methods in accordance with the invention.

In methods in accordance with the invention, the entire process of removing a component of interest, i.e., purifying, a raw biological sample takes place within a well in a microfluidic device. Since these methods do not require that the sample be introduced into the channels or chambers within the interior of the microfluidic device, the problems associated with flowing a raw sample through those channels or chambers are completely eliminated. Nevertheless, since the well is connected to the network of microfluidic channels in the device, the integration and automation provided by microfluidic technology can still be exploited.

The invention can be embodied in other specific forms without departing from the spirit or essential characteristics thereof. The present embodiments, therefore, are to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A method of purifying a biological component found in a biological sample by extracting said biological component from said biological sample, said method being performed in a microfluidic device having at least one well for receiving said biological sample and at least one channel for introducing and removing fluids in performing the present method, said method comprising providing a plurality of magnetic beads having a factor with an affinity for said biological component, introducing said magnetic beads into said well together with said biological sample, manipulating said biological sample to release said biological component in proximity to said magnetic beads, magnetically segregating said magnetic beads within said well, removing said biological sample from said well, introducing an elution solution for said biological component into said well and removing said elution solution together with said biological component from said well.

2. The method of claim 1 wherein said biological sample is washed from said magnetic beads coated with said biological component prior to the introduction of said elution solution.

3. The method of claim 1 wherein said factor and said biological component comprise DNA.

4. The method of claim 1 wherein a magnet applied to the exterior of said well causes said magnetic beads to segregate to a side wall of said well.

5. The method of claim 1 wherein said biological sample is removed from said well after said biological sample has been manipulated to release said biological component by introducing and removing a wash liquid through said well.

6. The method of claim 5 wherein said magnetic beads are agitated when said wash liquid is in contact with said magnetic beads.

7. The method of claim 1 wherein said magnetic beads are agitated when said elution solution is in contact with said magnetic beads.

8. The method of claim 1 wherein said magnetic beads are magnetically manipulated to aggregate proximate said at least one channel used to introduce said elution solution during the practice of said method when said elution solution is fed into said well.

9. The method of claim 8 wherein said magnetic beads are magnetically manipulated to be removed from an area proximate said at least one channel when said elution solution is removed from said well through said at least one channel.

10. The method of claim 1 wherein an electric field is applied to said magnetic beads when said elution solution is in contact with said magnetic beads.

11. A method of purifying a biological component found in a biological sample by extracting said biological component from said biological sample, said method being performed in a microfluidic device having a well for receiving said biological sample and a channel for introducing and withdrawing fluids into and from said well, said method comprising providing a plurality of magnetic beads having a factor with an affinity for said biological component, introducing said magnetic beads to said well together with said biological sample, manipulating said biological sample to release said biological component in proximity to said magnetic beads, magnetically segregating said magnetic beads proximate said channel, introducing a wash liquid to said well through said channel for washing said biological sample from said magnetic beads and maintaining a volume of wash liquid between said magnetic beads and said biological sample, introducing an elution solution for said biological component from said channel, said elution solution residing in proximity to said wash liquid and spaced from said biological sample, and removing said elution solution and biological component from said well through said channel.

12. The method of claim 11 wherein said factor and said biological component comprise DNA.

13. The method of claim 11 wherein a magnetic force applied to the exterior of said well segregates said magnetic beads to a sidewall of said well.

14. The method of claim 11 wherein said magnetic beads are agitated when said elution solution is in contact with said magnetic beads.

15. The method of claim 11 wherein said magnetic beads are magnetically manipulated to be removed from an area proximate said channel when said elution solution is removed from said well through said channel.

16. A method of purifying a biological component found in a biological sample by extracting said biological component from said biological sample, said method being performed in a microfluidic device comprising a well for receiving said biological sample and a first and a second channel for introducing and withdrawing fluids into and from said well, said method comprising providing a plurality of magnetic beads having a factor with an affinity for said biological component, introducing said magnetic beads to said well together with said biological sample, manipulating said biological sample to release said biological component in proximity to said magnetic beads, magnetically segregating said magnetic beads proximate said first channel, introducing a wash liquid through said first channel and drawing said wash liquid and biological sample from said well through said second channel, introducing an elution solution for said biological sample through said first channel in proximity to said magnetic beads, and removing said elution solution together with said biological component from said well through said first channel.

17. The method of claim 16 wherein said factor and said biological component comprise DNA.
